(19) United States
(12) Patent Application Publication
(10) Pub. No.: US 2007/0027399 A1
(43) Pub. Date: Feb. 1, 2007

(54) System using a bluetooth headphone for heartbeat monitoring, electronic recording, and displaying data

(76) Inventor: Hsien-Lung Chou, Taichung City (TW)

Correspondence Address: EGBERT LAW OFFICES, 412 MAIN STREET, 7TH FLOOR, HOUSTON, TX, 77002, US

(21) Appl. No.: 11/490,727

(22) Filed: Jul. 21, 2006

(30) Foreign Application Priority Data
Jul. 29, 2005 (TW) 094212987

(51) Int. Cl. A61B 5/04

(52) U.S. Cl. 600/523

(57) ABSTRACT

The present invention provides a heartbeat monitoring and electronic recording or display device using bluetooth headphone comprises: a headphone, which has a heartbeat detecting module and an algorithm and control unit, and among them, the heartbeat detecting module measures the pulse by the ear, and transmit the signal to the algorithm and control unit, and the algorithm and control unit calculates the number of the beats using application program/sport information integrated system, and coverts the data into signals; an electronic device that has a recording or display unit to display the data for the user to read and use; a bluetooth module, which transmits the heartbeat signal between the headphone and the electronic device using wireless transmitting method; and by so doing, the heartbeat detecting module continue to detect the pulse and transmit the signal to the algorithm and control unit, and calculate, process, convert the data collected, and the bluetooth module transmit the signal to the electronic device to record and display its data, and by so doing, the data is detected by the headphone, and is transmitted with wireless device.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

The present invention relates generally to a system for heartbeat measuring and transmitting data, and more particularly to an electronic device that has a display to record or to display the data after calculating, processing and converting the pulse signals gathered from an headphone using bluetooth wireless transmitting technology.

### BACKGROUND OF THE INVENTION

Because of today's lifestyle, people are easily exhausted from busy work and lack of recreational activity. Realizing that their health is in jeopardy, they become more health conscious, and take physical fitness training more seriously.

However, so as to understand the limitation of their physical ability, how to do the exercise safely, and how much time to spend exercising, a tool is needed to record all the activities for coordinating the activities. This tool must have indicators for measuring the amount of exercise as well as measuring the activity of the primary organ of human body so as to help the user understand its physical condition. The information can determine whether or not the scope of exercise is safe and whether or not the time for exercise is safe. This type of equipment generally comprises measuring indicators for body temperature, heartbeat, calories, body fat, step counting, blood pressure, and blood sugar.

In terms of measuring the number of heartbeats, this heart rate information can be used to evaluate physical condition of the human body and to determine the cardiopulmonary function as a strength indicator. The heart rate can also estimate energy expenditure as well as the training result. Therefore, the heartbeat is an important physiological indicator for human body. The measuring of heartbeats comprises resting heart rate, exercise heart rate, and recovery heart rate after the exercise.

The resting heart rate means the number of heartbeats per minute after the human heart is resting for a long time, which is usually based on the testing measured when people first awaken in the morning before getting out of bed. Generally, the lower the resting heart rate, the better the cardiopulmonary function is. On the other hand, the measuring of the exercise heart beat is harder because of the physical activity. It often needs special heart rate measuring instruments, and the measuring can only be based on the heart rate measured right after the exercise. The exercise heart rate can reflect the stimulation the exercise had on the body, and under the special condition of exercise strength, the lower the exercise heart rate, the better the cardiopulmonary function is. As for the recovery heart rate, which is one of the indicators of cardiopulmonary function, it is based on the heart beat during the time the exercise heart rate recovers to the resting heart rate, and the faster the heart rate recover after exercise, the better the cardiopulmonary function is.

Conventional heartbeat measuring structures comprise digital watches and treadmills. Among them, the digital watch places the measuring unit on the chest, and the measuring unit transmits a signal to the digital watch through a wire transmission to display the data on the screen. For treadmill, both hands are placed on the handle of the treadmill, and the information is transmitted through the signal to the display on the control panel using a detective device to show the data. Even though the conventional structure mentioned above can measure cardiopulmonary function, the signal transmitted by wire is not convenient while exercising. Moreover, holding the handle of the treadmill while running does not meet the design for human factors engineering, and the data collected may not be accurate.

Thus, to overcome the aforementioned problems of the prior art, it would be an advancement in the art to provide an improved structure that can significantly improve efficacy.

To this end, the inventor has provided the present invention of practicability after deliberate design and evaluation based on years of experience in the production, development and design of related products.

### BRIEF SUMMARY OF THE INVENTION

The system using a bluetooth headphone for heartbeat monitoring, electronic recording, and displaying data of the present invention has the following features, effects, and structures.

The present invention detects the pulse signal by the ear, and effectively utilizes a portable electronic device using bluetooth wireless transmitting methods. It simplifies the structure, and helps the user evaluate and monitor the heartbeat effectively.

Using the bluetooth transmitting method with all types of electronic devices, a suitable electronic device can be selected to test the heartbeat whether they are active or inactive. At the same time, transmitting speed is fast and not blocked by objects or by distance, which is much more convenient.

The application program, such as for sports information, is an integrated system placed on an algorithm and control unit 12. A microprocessing unit 21 processes, calculates, converts signal and data, which is more convenient for recording or outputting to the display unit 22 for display and use.

Using the headphone with a heartbeat detecting module 11, the algorithm and control unit 12 as well as a signal emission unit 31 process, calculate, and convert, making the system easy to use. Use with any electronic device that is compatible with the signals increases its practicability.

Although the invention has been explained in relation to its preferred embodiment, it is to be understood that many other possible modifications and variations can be made without departing from the spirit and scope of the invention as hereinafter claimed.

### BRIEF DESCRIPTION OF THE SEVERAL VIEWS OF THE DRAWINGS

FIG. 1 shows a schematic view of the preferred embodiment of the present invention.

FIG. 2 shows a perspective view of the operation of a preferred embodiment of the present invention.

FIG. 3 shows another schematic view of the preferred embodiment of the present invention.

FIG. 4 shows a perspective view of the operation of another preferred embodiment of the present invention.

FIG. 5 shows a perspective view of the operation of still another preferred embodiment of the present invention.

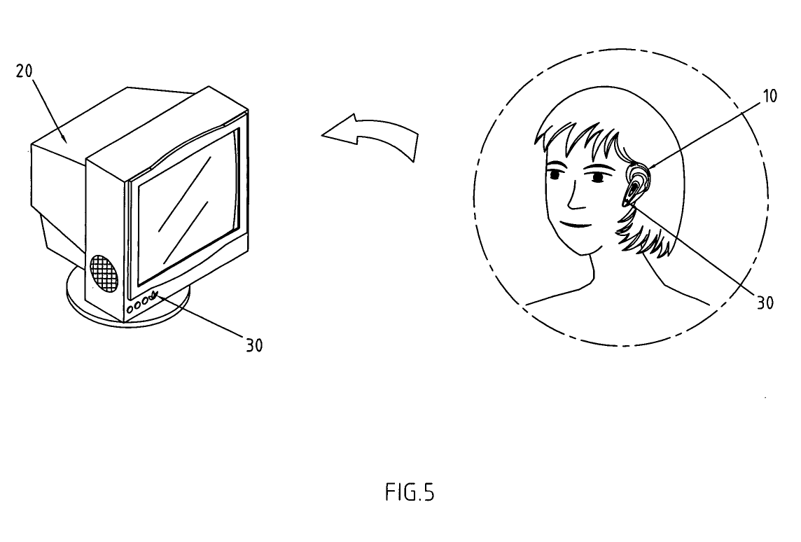

FIG. 6 shows a perspective view of the operation of yet another preferred embodiment of the present invention.

### DETAILED DESCRIPTION OF THE INVENTION

The features and the advantages of the present invention will be more readily understood upon a thoughtful deliberation of the following detailed description of a preferred embodiment of the present invention with reference to the accompanying drawings.

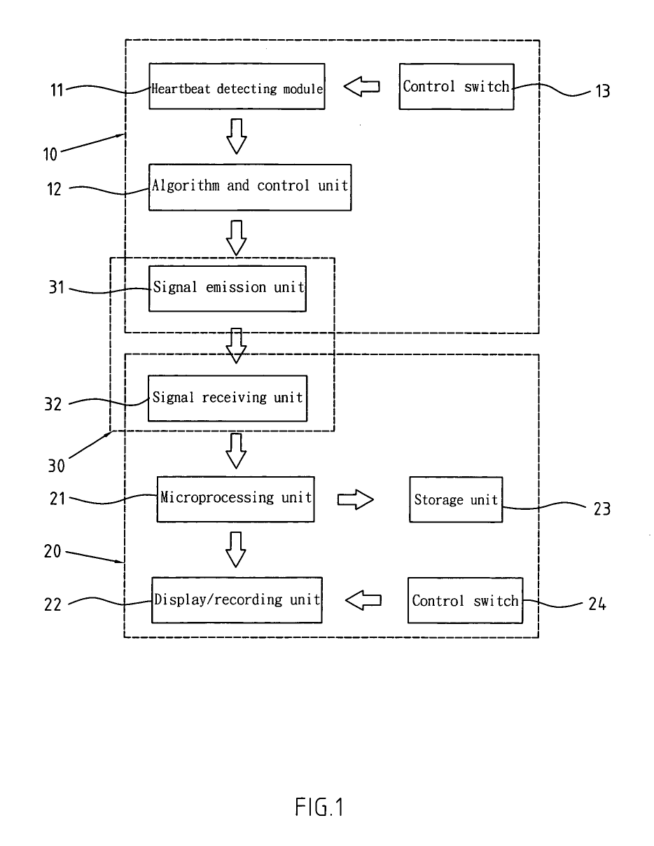

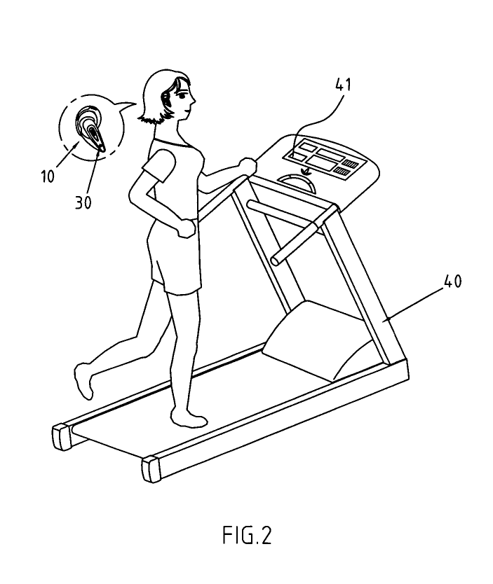

As shown in FIGS. 1 and 2, there is the system using a bluetooth headphone for heartbeat monitoring, electronic recording, and displaying data.

The invention includes a headphone 10, an electronic device 20, and a bluetooth module 20 as a transmitter for the two devices mentioned above.

The headphone 10 has a heartbeat detecting module 11 and an algorithm and control unit 12. The heartbeat detecting module 11 measures the pulse by the ear and transmits the signal to the algorithm and control unit 12. The algorithm and control unit 12 calculates the number of the beats and coverts the data into signals.

A control switch 13 controls the on and off of the heartbeat detecting module 12.

An electronic device 20, being attached to fitness equipment 40 with a control panel 41, has a microprocessing unit 21 and a display unit. The microprocessing unit 21 sends the data to the display unit 22 or recording unit (not shown) for reading or using with an application program, such as a sport information integrated system. The unit circuits to convert, calculate and process the signal.

A storage unit 23 stores data calculated, processed and converted by the microprocessing unit. A control switch 24 controls the on and off of the display unit that shows the heartbeat data.

A bluetooth module 30 transmits the heartbeat signal between the headphone 10 and the electronic device 20 using a wireless transmitting method. The bluetooth module 30 has a signal emission unit 31 that is placed inside the headphone 20, and it sends out the signal coming from the algorithm and control unit 12.

A signal receiving unit 32 is placed inside the electronic device 20 to send and receive signals and to transmit the signals to the display unit 22 or a recording unit or a device with both a recording and a display unit. The signal emission unit 31 and the signal receiving unit 32 respond to each other by an identification code.

Through the above structure and design, the heartbeat detecting module 11 continues to detect the pulse and transmit the signals to the algorithm and control unit 12. The unit 12 calculates, processes, and can convert the data collected. The bluetooth module 30 transmits the signal to any electronic device 20 of any manager device that has the correct application program or sport information integrated system to show the data. By so doing, the data is detected by the headphone 10 and is transmitted by the wireless device.

It is worth mentioning that because the bluetooth wireless transmitting technology mainly uses a Personal Area Network with wireless electronic interface and transmits the data and signal in actual time, the transmitting speed is not blocked by a wall, pocket, or briefcase. Any portable electronic device with bluetooth function can be connected. Moreover, the entire structure of the bluetooth module can easily and simply be installed on any electronic device.

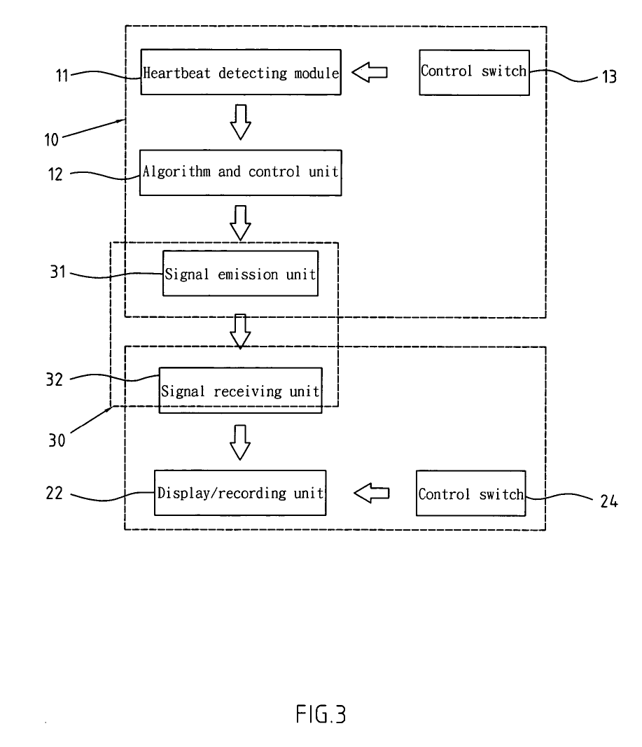

Another preferred embodiment of the present invention, as shown in FIG. 3, has an application program, such as a sport information integrated system, placed in the algorithm and control unit 12 to covert, calculate, and process the digital signal. The electronic device 20 does not need an extra microprocessing unit 21, which simplifies the structure and reduce cost.

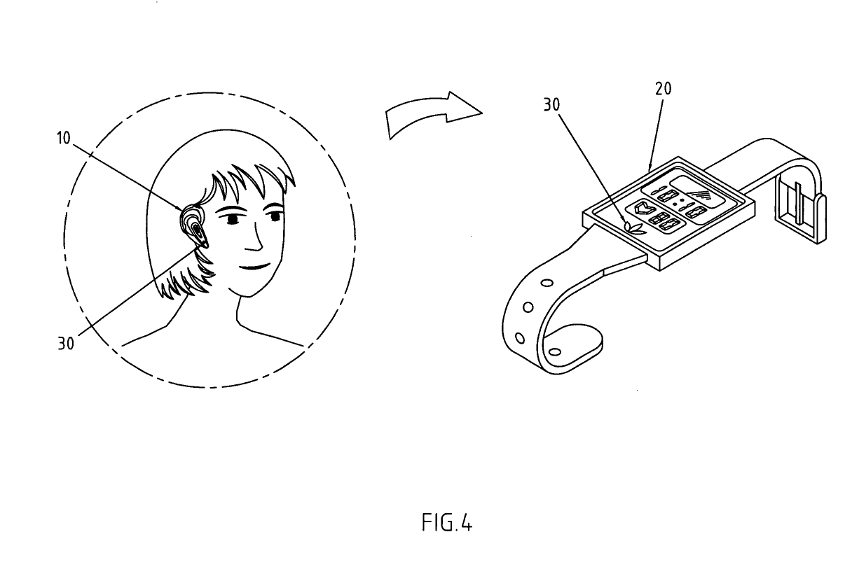

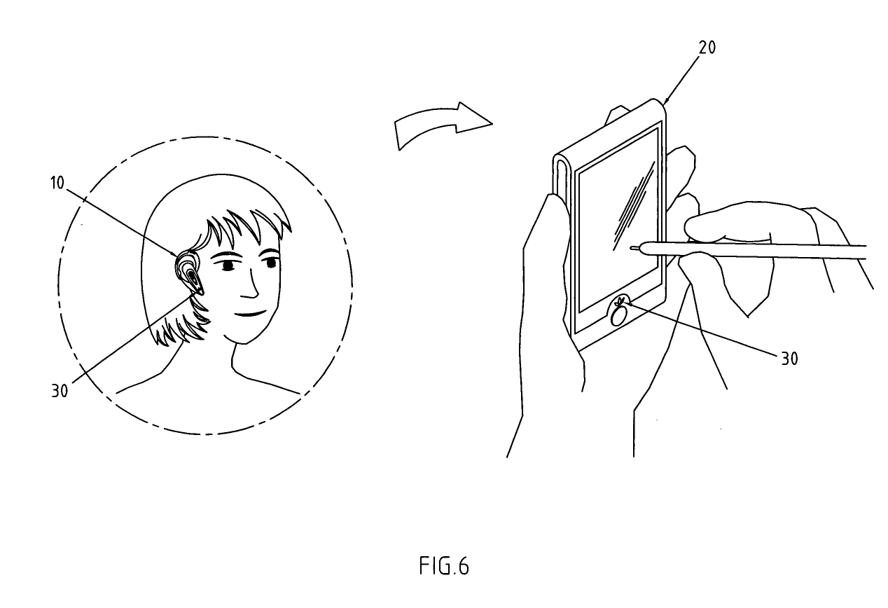

As shown in FIGS. 4 and 6, the present invention also uses other electronic devices 20, such as a digital watch, PDA, and PC, having a manager device with the application program or sport information integrated system. Thus, the suitable electronic device 20 can be selected according the situations, such as during ball games, biking, running, yoga, or reading and using computers.

I claim:

1. A system using a bluetooth headphone for heartbeat monitoring, electronic recording, and displaying data, said system comprising:
a headphone, having a heartbeat detecting module and an algorithm and control unit, said heartbeat detecting module being able to measure pulse by an ear of a user and to transmit signals to said algorithm and control unit, said algorithm and control unit being able to calculate a number of beats and to convert data into signals;
an electronic device, having a microprocessing unit or a recording unit or a display unit, said microprocessing unit sending data to said recording unit or said display unit, after using an application program/sport information integrated system and circuits to convert, calculate and process the signal;
a bluetooth module, transmitting a heartbeat signal between said headphone and said electronic device using a wireless transmitting method; and
a signal receiving unit, being placed inside said electronic device to send and receive signals before transmitting the signal to said recording or said display unit.

2. The system defined in claim 1, wherein said heartbeat detecting module continues to detect the pulse and transmit the signal to the algorithm and control unit, and continues to calculate, process, convert the data collected, and wherein said bluetooth module transmits the signal to any electronic device of manager device that has application program/sport information integrated system to show its data, the data being detected by the headphone and transmitted by a wireless device.

3. A system using a bluetooth headphone for heartbeat monitoring, electronic recording, and displaying data, said system comprising:
a headphone, having a heartbeat detecting module and an algorithm and control unit, said heartbeat detecting module being able to measure pulse by an ear of a user and to transmit signals to said algorithm and control unit, said algorithm and control unit being able to calculate a number of beats using an application program/sport information integrated system and to convert data into signals;
an electronic device, having a display unit or a recording unit displaying data;
a bluetooth module, transmitting a heartbeat signal between said headphone and said electronic device using a wireless transmitting method, said bluetooth module being further comprised of a signal emission unit placed inside said headphone and sending out a signal coming from said algorithm and control unit; and
a signal receiving unit, being placed inside said electronic device to send and receive signals before transmitting data to said recording unit or said display unit.

4. The system defined in claim 1, wherein said electronic device has a bluetooth module, being wearable as a digital watch with a display unit.

5. The system defined in claim 1, wherein said electronic device has a bluetooth module, being placed in a portable laptop or Personal Digital Assistant.

6. The system defined in claim 1, wherein said electronic device is comprised of a personal computer having a bluetooth module.

7. The system defined in claim 1, wherein said electronic device is comprised of fitness equipment having a bluetooth module.

8. The system defined in claim 1, wherein said electronic device has a storage unit, storing data calculated, processed and converted by said microprocessing unit.

9. The system defined in claim 1, wherein said headphone comprises a control switch, controlling an on and off switch of said heartbeat detecting module.

10. The system defined in claim 1, wherein said electronic device comprises a control switch, controlling an on and off switch of said display unit showing heartbeat data.

11. The system defined in claim 1, wherein said recording unit or said display unit on said electronic device is a recording unit or a display unit or a device with both recording unit and display unit.

12. A system using a bluetooth headphone for heartbeat monitoring, electronic recording, and displaying data, said system comprising:
a headphone, having a heartbeat detecting module and an algorithm and control unit, said heartbeat detecting module being able to measure pulse by an ear of a user and to transmit signals to said algorithm and control unit, said algorithm and control unit being able to calculate a number of beats using an application program/sport information integrated system and to convert data into signals; and
a bluetooth module, transmitting a heartbeat signal between said headphone and said electronic device using a wireless transmitting method, said bluetooth module being further comprised of a signal emission unit placed inside said headphone and sending out a signal coming from said algorithm and control unit.
